(19) United States
(12) Patent Application Publication
(10) Pub. No.: US 2021/0130445 A1
(43) Pub. Date: May 6, 2021

(54) CGRP Antagonists and Botulinum Toxins for the Treatment of Inflammatory and Neurologic Disorders

(71) Applicant: Allergan Pharmaceuticals International Limited, Dublin 17 (IE)

(72) Inventor: Mitchell F. Brin, Newport Beach, CA (US)

(21) Appl. No.: 16/921,611

(22) Filed: Jul. 6, 2020

Related U.S. Application Data
(60) Provisional application No. 62/870,931, filed on Jul. 5, 2019.

(51) Int. Cl. C07K 16/18; A61P 21/00; A61K 38/48; A61K 45/06

(52) U.S. Cl. CPC C07K 16/18; A61K 45/06; A61K 38/4893; A61P 21/00

(57) ABSTRACT

The application is related to the treatment of diseases and disorders associated with pain by the administration of a CGRP-antagonist and a clostridial derivative.

### FIELD

The application is related to medicaments and methods for the treatment of diseases and disorders associated with pain by the administration of a CGRP-antagonist to a patient who is undergoing treatment with a clostridial derivative. The present application is directed to a medicament functioning as a calcitonin-gene-related-peptide (hereafter referred to as CGRP) antagonist administered in combination with a clostridial derivative, such as a botulinum toxin, for treating pain and related disorders.

### BACKGROUND

Therapeutic treatment of chronic or acute pain is among the most common reasons patients seek medical care. Chronic pain may be particularly disabling, and the cumulative economic impact of chronic pain is huge. A large portion of the population that is over the age of 65 may suffer from any of a variety of health issues which can predispose them to chronic or acute pain. An even greater portion of the nursing home population may suffer from chronic pain. (U.S. Pat. No. 10,314,739)

Current treatments for chronic pain may include pharmaceutical analgesics and electrical neurostimulation. While both these techniques may provide some level of relief, they can have significant drawbacks. For example, pharmaceuticals may have a wide range of systemic side effects, including gastrointestinal bleeding, interactions with other drugs, and the like. Opioid analgesics can be addictive and may also of themselves be debilitating. The analgesic effects provided by pharmaceuticals may be relatively transient, making them cost-prohibitive for the aging population that suffers from chronic pain. While neurostimulators may be useful for specific applications, they generally involve surgical implantation, an expensive procedure which carries its own risks, side effects, contraindications, on-going maintenance issues, and the like.

In general, it would be advantageous to provide improved methods for management of chronic and/or acute pain.

### SUMMARY

The application provides methods for treating, preventing, alleviating or reducing the frequency of occurrence of pain perceived by a patient by the use antagonists of calcitonin gene-related peptide (CGRP-antagonists), wherein the patient is concurrently undergoing treatment with a clostridial derivative. In some embodiments, the patient is administered CGRP-antagonist, and, a clostridial derivative. In some embodiments, the clostridial derivative is a recombinant clostridial toxin, a recombinant modified clostridial toxin, fragments of botulinum toxin, or targeted exocytosis modulators (TEMs), or combinations thereof. In some embodiments, the CGRP-antagonist is ubrogepant, atogepant, or a pharmaceutically acceptable salt, ester or prodrug thereof. In some embodiments, the CGRP-antagonist is an antibody is selected from galcanezumab, fremanezumab, eptinezumab, and erenumab.

### DESCRIPTION

The application provides methods for treating, preventing, alleviating or reducing the frequency of occurrence of pain in patients by the use antagonists of calcitonin gene-related peptide (CGRP), wherein the patient is concurrently undergoing treatment with a clostridial derivative.

In some embodiments, the application provides methods for the treatment of pain, wherein the pain is other than pain associated with headache or migraine. In a preferred embodiment, the pain is neuropathic type pain or inflammatory type pain. In some embodiments, the pain is neuralgia, preferably trigeminal neuralgia.

In some embodiments, the pain is caused by diabetes mellitus type I or II, viral or retroviral infection, cancer chemotherapy, radiotherapy, a surgical procedure, alcoholism, facial neuralgia, trauma, radiculopathy or radiculagia, cruralgia or thoracic outlet syndrome, fibromyalgia and restless leg syndrome.

In some embodiments, the pain is selected from acute and chronic inflammatory demyelinating polyradiculoneuropathy, alcoholic polyneuropathy, chemotherapy-induced polyneuropathy, complex regional pain syndrome, an entrapment neuropathy; HIV sensory neuropathy, an iatrogenic neuralgia, idiopathic sensory neuropathy, nerve compression or infiltration by a tumor, nutritional deficiency-related neuropathies, painful diabetic neuropathy, phantom limb pain, postherpetic neuralgia, postradiation plexopathy, radiculopathy, toxic exposure-related neuropathies, tic douloureux (trigeminal neuralgia), and/or posttraumatic neuralgias.

In some embodiments, the pain is selected from post-surgical pain, post-operative pain, dental pain, pain associated with burn, wound or kidney stone, pain associated with trauma, traumatic head injury, pain associated with musculo-skeletal disorders, rheumatoid arthritis, osteoarthritis, visceral pain, colitis, pancreatitis, gastritis, ankylosing spondylitis, sero-negative (non-rheumatoid) arthropathies, non-articular rheumatism and peri-articular disorders, and pain associated with cancer, pain associated with sickle-cell anemia, peripheral neuropathy, post-herpetic neuralgia, trigeminal neuralgia, general neuralgia, intercostal neuralgia, glossopharyngeal neuralgia, postherpetic neuralgia.

In some embodiments, the pain is selected from rheumatic pain, pain associated with mucositis, and dysmenorrhea, post-surgical pain and/or cancer pain, pain associated with rheumatoid arthritis or pain associated with osteoarthritis.

In some embodiments, the pain is associated with diabetic vasculopathy, diabetic retinopathy or diabetic symptoms associated with insulitis.

In some embodiments, the said pain is acute pain, chronic pain, neuropathic pain, inflammatory pain, visceral pain, nociceptive pain, pain associated with multiple sclerosis, pain associated with irritable bowel syndrome or inflammatory bowel disease, pain associated with dysmenorrhea, pelvic pain, pain associated with cystitis, pain associated with pancreatitis, pain associated with Crohn's disease, pain associated with epilepsy or an epileptic condition, visceral pain, radicular pain, sciatica, back pain, neck pain, severe or intractable pain, breakthrough pain, postsurgical pain, stroke, cancer pain, seizure disorder, causalgia and/or chemo-induced pain. In some embodiments, the pain is pain is acute pain. In some embodiments, the pain is chronic pain.

In some embodiments, the clostridial derivative is a botulinum toxin. In one embodiment, the clostridial derivative is onabotulinumtoxinA. In some embodiments, the methods relate to administering to the patient a CGRP-antagonist, and a botulinum toxin, preferably onabotulinumtoxinA. In embodiments, the combination therapy of botulinum toxin and a CGRP antagonist reduces the frequency, severity and/or duration of pain in patients in need thereof.

In some embodiments, the CGRP-antagonist is an anti-calcitonin gene-related peptide receptor antibody (anti-CGRP antibody) or antigen-binding fragment thereof. For example, the antibody can be selected from galcanezumab, fremanezumab, eptinezumab or erenumab. In some embodiments, the anti-CGRP antibody or fragment thereof is administered at a dosage that is about 20% or 30% or 40% or 50% or 60% or 70% or 80% lower than the recommended dosage for the anti-CGRP antibody monotherapy. In some embodiments, the anti-CGRP antibody or antigen-binding fragment thereof is administered to a peripheral nerve, a cranial nerve, or combinations thereof.

For example, erenumab can be administered weekly, biweekly, monthly, every two months, every three months, every four months, every five months or every six months at a dosage of about 5 mg to about 500 mg.

Erenumab can be administered parenterally, subcutaneously or by peripheral administration. (Brauser D., Phase 3 STRIVE and ARISE Trials Show Efficacy, Safety for Erenumab in Migraine Prevention, Medscape Medical News, 2017).

In some embodiments, erenumab can be administered to the patient over the course of a set treatment period. (U.S. Patent Publication No. 20160311913) The treatment period can begin upon administration of a first dose of erenumab and ends upon administration of a final dose of erenumab. The combination therapy with botulinum toxin includes administration of botulinum toxin prior to, during or after the treatment period with erenumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In some embodiment, erenumab is administered in a pharmaceutical composition comprising a buffer (preferably an acetate buffer), a surfactant (preferably polysorbate 20 or polysorbate 80), and a stabilizing agent (preferably sucrose). In one particular embodiment, the treatment period is at least about 6 months and produces a statistically significant reduction in the frequency, duration, or severity of chronic pain in the patient as compared to patients treated with erenumab or botulinum toxin alone.

In one embodiment, erenumab can be administered subcutaneously at a dose of about 5 mg to about 500 mg every one, two, three, four, five, six, seven, eight, nine or ten weeks.

In one embodiment, erenumab can be administered subcutaneously at a dose of about 10 mg to about 200 mg every one, two, three, four, five, six, seven, eight, nine or ten weeks.

In one embodiment, erenumab can be administered subcutaneously at a dose of about 25 mg to about 150 mg every one, two, three, four, five, six, seven, eight, nine or ten weeks.

In one embodiment, erenumab can be administered subcutaneously at a dose of about 90 mg to about 120 mg every one, two, three, four, five, six, seven, eight, nine or ten weeks.

In one embodiment, erenumab can be administered subcutaneously at a dose of about 50 mg to about 60 mg every one, two, three, four, five, six, seven, eight, nine or ten weeks.

In one embodiment, erenumab can be administered subcutaneously at a dose of about 70 mg every one, two, three, four, five, six, seven, eight, nine or ten weeks.

In one embodiment, erenumab can be administered subcutaneously at a dose of about 140 mg every one, two, three, four, five, six, seven, eight, nine or ten weeks.

In one embodiment, erenumab can be administered subcutaneously at a monthly dose of about 140 mg.

In one embodiment, erenumab can be administered subcutaneously at a monthly dose of about 70 mg.

In one embodiment, erenumab can be administered subcutaneously at a dose of about 140 mg every two months.

In one embodiment, erenumab can be administered subcutaneously at a dose of about 70 mg every two months.

In one embodiment, erenumab can be administered subcutaneously at a dose of about 140 mg every three months.

In one embodiment, erenumab can be administered subcutaneously at a dose of about 70 mg every three months.

In one embodiment, an anti-CGRP antibody galcanezumab can be administered weekly, biweekly, monthly, every two months, every three months, every four months, every five months or every six months at a dosage of about 5 mg to about 500 mg.

In some embodiments, galcanezumab can be administered to the patient over the course of a set treatment period. The treatment period can begin upon administration of a first dose galcanezumab and ends upon administration of a final dose of galcanezumab. The combination therapy with botulinum toxin includes administration of botulinum toxin prior to, during or after the treatment period with galcanezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with galcanezumab or botulinum toxin alone.

In one embodiment, galcanezumab is administered subcutaneously at a dose of about 10 mg to about 500 mg every one, two, three, four, five, six, seven, eight, nine or ten weeks.

In one embodiment, galcanezumab is administered subcutaneously at a dose of about 50 mg to about 300 mg every one, two, three, four, five, six, seven, eight, nine or ten weeks.

In one embodiment, galcanezumab is administered subcutaneously at a dose of about 75 mg to about 250 mg every one, two, three, four, five, six, seven, eight, nine or ten weeks.

In one embodiment, galcanezumab is administered subcutaneously at a dose of about 75 mg to about 100 mg every one, two, three, four, five, six, seven, eight, nine or ten weeks.

In one embodiment, galcanezumab is administered subcutaneously at a dose of about 150 mg to about 220 mg every one, two, three, four, five, six, seven, eight, nine or ten weeks.

In one embodiment, galcanezumab is administered subcutaneously at a dose of about 120 mg every one, two, three, four, five, six, seven, eight, nine or ten weeks.

In one embodiment, galcanezumab is administered subcutaneously at a dose of about 240 mg every one, two, three, four, five, six, seven, eight, nine or ten weeks.

In one embodiment, galcanezumab is administered subcutaneously at a monthly dose of about 240 mg.

In one embodiment, galcanezumab is administered subcutaneously at a monthly dose of about 120 mg.

In one embodiment, galcanezumab is administered subcutaneously at a dose of about 240 mg every two months.

In one embodiment, galcanezumab is administered subcutaneously at a dose of about 120 mg every two months.

In one embodiment, galcanezumab is administered subcutaneously at a dose of about 240 mg every three months.

In one embodiment, galcanezumab is administered subcutaneously at a dose of about 120 mg every three months.

In some embodiments, fremanezumab can be administered to the patient over the course of a set treatment period. (Silberstein, S. D., et. al., N Engl J Med 2017; 377:2113-22.) The treatment period can begin upon administration of a first dose fremanezumab and ends upon administration of a final dose of fremanezumab. The combination therapy with botulinum toxin includes administration of botulinum toxin prior to, during or after the treatment period with fremanezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with fremanezumab or botulinum toxin alone.

In one embodiment, fremanezumab is administered subcutaneously at a dose of about 100 mg to about 1000 mg every one, two, three, four, five, six, seven, eight, nine or ten weeks.

In one embodiment, fremanezumab is administered subcutaneously at a dose of about 150 mg to about 700 mg every one, two, three, four, five, six, seven, eight, nine or ten weeks.

In one embodiment, fremanezumab is administered subcutaneously at a dose of about 150 mg to about 500 mg every one, two, three, four, five, six, seven, eight, nine or ten weeks.

In one embodiment, fremanezumab is administered subcutaneously at a dose of about 150 mg to about 200 mg every one, two, three, four, five, six, seven, eight, nine or ten weeks.

In one embodiment, fremanezumab is administered subcutaneously at a dose of about 150 mg to about 500 mg every one, two, three, four, five, six, seven, eight, nine or ten weeks.

In one embodiment, fremanezumab is administered subcutaneously at a dose of about 225 mg every one, two, three, four, five, six, seven, eight, nine or ten weeks.

In one embodiment, fremanezumab is administered subcutaneously at a dose of about 450 mg every one, two, three, four, five, six, seven, eight, nine or ten weeks.

In one embodiment, fremanezumab is administered subcutaneously at a dose of about 675 mg every one, two, three, four, five, six, seven, eight, nine or ten weeks.

In one embodiment, fremanezumab is administered subcutaneously at a monthly dose of about 225 mg.

In one embodiment, fremanezumab is administered subcutaneously at a monthly dose of about 450 mg.

In one embodiment, fremanezumab is administered subcutaneously at a monthly dose of about 675 mg.

In one embodiment, fremanezumab is administered subcutaneously at a dose of about 225 mg every two months.

In one embodiment, fremanezumab is administered subcutaneously at a dose of about 450 mg every two months.

In one embodiment, fremanezumab is administered subcutaneously at a dose of about 225 mg every three months.

In one embodiment, fremanezumab is administered subcutaneously at a dose of about 450 mg every three months.

In one embodiment, fremanezumab is administered subcutaneously at a dose of about 675 mg every three months.

In some embodiments, eptinezumab can be administered to the patient over the course of a set treatment period. The treatment period can begin upon administration of a first dose eptinezumab and ends upon administration of a final dose of eptinezumab. The combination therapy with botulinum toxin includes administration of botulinum toxin prior to, during or after the treatment period with eptinezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with eptinezumab or botulinum toxin alone.

In one embodiment, eptinezumab is administered subcutaneously at a dose of about 50 mg to about 1000 mg every one, two, three, four, five, six, seven, eight, nine or ten weeks.

In one embodiment, eptinezumab is administered subcutaneously at a dose of about 100 mg to about 700 mg every one, two, three, four, five, six, seven, eight, nine or ten weeks.

In one embodiment, eptinezumab is administered subcutaneously at a dose of about 200 mg to about 500 mg every one, two, three, four, five, six, seven, eight, nine or ten weeks.

In one embodiment, eptinezumab is administered subcutaneously at a dose of about 250 mg to about 350 mg every one, two, three, four, five, six, seven, eight, nine or ten weeks.

In one embodiment, eptinezumab is administered subcutaneously at a dose of about 300 mg every one, two, three, four, five, six, seven, eight, nine or ten weeks.

In one embodiment, eptinezumab is administered subcutaneously at a monthly dose of about 100 mg.

In one embodiment, eptinezumab is administered subcutaneously at a monthly dose of about 200 mg.

In one embodiment, eptinezumab is administered subcutaneously at a monthly dose of about 300 mg.

In one embodiment, eptinezumab is administered subcutaneously at a dose of about 100 mg every two months.

In one embodiment, eptinezumab is administered subcutaneously at a dose of about 200 mg every two months.

In one embodiment, eptinezumab is administered subcutaneously at a dose of about 300 mg every two months.

In one embodiment, eptinezumab is administered subcutaneously at a dose of about 100 mg every three months.

In one embodiment, eptinezumab is administered subcutaneously at a dose of about 200 mg every three months.

In one embodiment, eptinezumab is administered subcutaneously at a dose of about 300 mg every three months.

In some embodiments, an antagonist of CGRP receptor can be administered in combination with a botulinum toxin. Preferably, the CGRP antagonist is selected from ubrogepant, atogepant, rimegepant or a pharmaceutically acceptable salt thereof.

In some embodiments, ubrogepant can be administered to the patient over the course of a set treatment period or indefinitely. The treatment period can begin upon administration of a first dose of ubrogepant and continue until the patient is administered ubrogepant on a regular or intermittent basis. The combination therapy with botulinum toxin includes administration of botulinum toxin prior to, during or after the treatment period with ubrogepant. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with ubrogepant or botulinum toxin alone.

In some embodiments, ubrogepant is administered at an oral dose of about 5 to about 500 mg once, twice or three times a day.

In some embodiments, ubrogepant is administered at an oral dose of about 25 mg once, twice or three times a day.

In some embodiments, ubrogepant is administered at an oral dose of about 50 mg once, twice or three times a day.

In some embodiments, ubrogepant is administered at an oral dose of about 100 mg once, twice or three times a day.

In some embodiments, ubrogepant is administered at an oral dose of about 200 mg once, twice or three times a day.

In some embodiments, atogepant can be administered to the patient over the course of a set treatment period or indefinitely. The treatment period can begin upon administration of a first dose atogepant and continue until the patient is administered atogepant on a regular or intermittent basis. The combination therapy with botulinum toxin includes administration of botulinum toxin prior to, during or after the treatment period with atogepant. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with atogepant or botulinum toxin alone.

In some embodiments, atogepant is administered at an oral dose of about 5 to about 500 mg once, twice or three times a day.

In some embodiments, atogepant is administered at an oral dose of about 25 mg once, twice or three times a day.

In some embodiments, atogepant is administered at an oral dose of about 50 mg once, twice or three times a day.

In some embodiments, atogepant is administered at an oral dose of about 100 mg once, twice or three times a day.

In some embodiments, atogepant is administered at an oral dose of about 200 mg once, twice or three times a day.

In some embodiments, rimegepant can be administered to the patient over the course of a set treatment period or indefinitely. The treatment period can begin upon administration of a first dose rimegepant and continue until the patient is administered rimegepant on a regular or intermittent basis. The combination therapy with botulinum toxin includes administration of botulinum toxin prior to, during or after the treatment period with rimegepant. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with rimegepant or botulinum toxin alone.

In some embodiments, rimegepant is administered at an oral dose of about 5 to about 500 mg once, twice or three times a day.

In some embodiments, rimegepant is administered at an oral dose of about 25 mg once, twice or three times a day.

In some embodiments, rimegepant is administered at an oral dose of about 50 mg once, twice or three times a day.

In some embodiments, rimegepant is administered at an oral dose of about 100 mg once, twice or three times a day.

In some embodiments, rimegepant is administered at an oral dose of about 200 mg once, twice or three times a day.

In some embodiments, rimegepant is administered at an oral dose of about 5 to about 500 mg once, twice or three times a day.

In some embodiments, rimegepant is administered at an oral dose of about 25 mg once, twice or three times a day.

In some embodiments, rimegepant is administered at an oral dose of about 50 mg once, twice or three times a day.

In some embodiments, rimegepant is administered at an oral dose of about 100 mg once, twice or three times a day.

In some embodiments, rimegepant is administered at an oral dose of about 200 mg once, twice or three times a day.

In some embodiments, the combination therapy with botulinum toxin and a CGRP antagonist reduces the frequency, severity and/or duration of pain in patients in need thereof.

In some embodiments, the combination therapy with CGRP-antagonists and botulinum toxin described herein is administered to a patient undergoing treatment with one or more additional medications for the treatment of pain. For example, morphine, codeine, hydrocodone, oxycodone, fentanyl, pethidine, methadone, pentazocine, sufentanil, levorphanol, dihydrocodeine, nalbuphine, butorphanol, tramadol, meptazinol, buprenorphine, dipipanone, alfentanil, remifentanil, oxymorphone, tapentadol, propoxyphene or hydromorphone. In one embodiment, the additional medication is acetaminophen, ibuprofen, ketaprofen, naproxen or aspirin. In one embodiment, the additional medication is a treatment for arthritis, for example, infliximab (Remicade), adalimumab (Humira), certolizumab pegol (Cimzia), golimumab (Simponi), or etanercept.

In some embodiments, the clostridial derivative is onabotulinumtoxinA and is administered at a dose of about 1 unit, about 2 units, about 3 units, about 4 units, about 5 units, about 6 units, about 7 units, about 8 units, about 9 units or about 10 units. The frequency of administration can be once every one, two, three, four, five, six, seven, eight, nine, ten, eleven, twelve, thirteen, fourteen, fifteen or sixteen weeks.

In some embodiments, onabotulinumtoxinA is administered at a dose of about 10 unit, about 15 units, about 20 units, about 25 units, about 30 units, about 40 units, about 45 units, about 50 units, about 55 units or about 60 units. The frequency of administration can be once every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about of about 25 unit, about 50 units, about 75 units, about 100 units, about 125 units, about 150 units, about 175 units, about 200 units, about 225 units or about 250 units every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about dose of about 1 to about 1,000 units. The frequency of administration can be once every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about 1 to about 100 units every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about 10 to about 50 units. The frequency of administration can be once every one, two, three, four, five, six, seven, eight, nine, ten, eleven, twelve, thirteen, fourteen, fifteen or sixteen weeks.

In some embodiments, onabotulinumtoxinA is administered parenterally.

In some embodiments, onabotulinumtoxinA is administered topically.

In some embodiments, onabotulinumtoxinA is administered subcutaneously or intramuscularly.

In some embodiments, onabotulinumtoxinA is administered intradermally.

In some embodiments, onabotulinumtoxinA is administered subcutaneously once every month or two months.

In some embodiments, onabotulinumtoxinA is administered at a dose of about 155 units.

The effective amount of the clostridial derivative can be measured in mass units (e.g. in ng or mg). The effective dose in weight units can be determined based on the intended effect.

For example, the effective weight can be determined based on the amount of clostridial derivative required to have a therapeutic effect on the muscle or a sensory effect. In some embodiments, the clostridial derivative can be administered at a dose of about 0.001 ng to about 1000 ng, preferably about 0.001 ng to about 500 ng, preferably about 0.01 ng to about 250 ng, preferably about 0.1 ng to about 150 ng, preferably about 1 ng to about 100 ng, preferably about 1 ng to about 10 ng. For example, onabotulinumtoxinA can be administered at a dose of about 1 ng, 2 ng, 3 ng, 4 ng, 5 ng, 6 ng, 7 ng, 8 ng, 9 ng or 10 ng.

In some embodiments, the CGRP-antagonist can be administered orally, sublingually, transdermally, subcutaneously, intravenously, intradermally or intramuscularly.

In one embodiment, the CGRP-antagonist can be administered intravenously. The intravenous formulation can contain a tonicity modifier to avoid crenation or hemolysis of red blood cells, and/or to mitigate or avoid pain and discomfort to the patient. Preferably, the formulation to be administered to the patient has an effective osmotic pressure that is approximately the same as that of the blood of the patient. Tonicity modifiers can be non-ionic tonicity modifiers such as glycerol, sorbitol, mannitol, sucrose, propylene glycol or dextrose, or a mixture thereof. Preferably the non-ionic tonicity modifier is dextrose, sucrose or mannitol, or a mixture thereof. Aqueous pharmaceutical formulations for intravenous administration generally can have a pH of from 3 to 9.

Stable liquid or solid pharmaceutical composition comprising a clostridial toxin derivative, a disaccharide, a surfactant and an antioxidant can be used in combination with CGRP-antagonists.

CGRP is a member of the calcitonin family of peptides, which in human exists in two form, α-CGRP and β-CGRP. α-CGRP and β-CGRP vary by three amino acids, have similar activities and exhibit differential distribution. At least two CGRP receptor subtypes may also account for differential activities. CGRP is produced in both peripheral and central neurons, and released by the trigeminal nerve. CGRP has been shown to be a potent vasodilator in the periphery, where CGRP-containing neurons are closely associated with blood vessels. CGRP-mediated vasodilatation is also associated with neurogenic inflammation, as part of a cascade of events that results in extravasation of plasma and vasodilation of the microvasculature and is present in migraine. cGRP is released by sensory nerves, e.g. the trigeminal nerve, which innervates part of the skin of the face. The trigeminal nerve has three major branches, a number of smaller branches and is the great sensory nerve of the head and neck, carrying touch, temperature, pain, and proprioception (position sense) signals from the face and scalp to the brainstem. Trigeminal sensory fibers originate in the skin, course toward the trigeminal ganglion (a sensory nerve cell body), pass through the trigeminal ganglion, and travel within the trigeminal nerve to the sensory nucleus of the trigeminal nerve located in the brainstem.

The three major branches of the trigeminal nerve are the ophthalmic (V₁, sensory), maxillary (V₂, sensory) and mandibular (V₃, motor and sensory) branches. The large trigeminal sensory root and smaller trigeminal motor root leave the brainstem at the midlateral surface of pons. The sensory root terminates in the largest of the cranial nerve nuclei which extends from the pons all the way down into the second cervical level of the spinal cord. The sensory root joins the trigeminal or semilunar ganglion between the layers of the dura mater in a depression on the floor of the middle crania fossa. The trigeminal motor root originates from cells located in the masticator motor nucleus of trigeminal nerve located in the midpons of the brainstem. The motor root passes through the trigeminal ganglion and combines with the corresponding sensory root to become the mandibular nerve. It is distributed to the muscles of mastication, the mylohyoid muscle and the anterior belly of the digastric. The three sensory branches of the trigeminal nerve emanate from the ganglia to form the three branches of the trigeminal nerve. The ophthalmic and maxillary branches travel in the wall of the cavernous sinus just prior to leaving the cranium. The ophthalmic branch travels through the superior orbital fissure and passes through the orbit to reach the skin of the forehead and top of the head. The maxillary nerve enters the cranium through the foramen rotundum via the pterygopalatine fossa. Its sensory branches reach the pterygopalatine fossa via the inferior orbital fissure (and supply sensation to the face, cheek and upper teeth) and pterygopalatine canal (and supply sensation to the soft and hard palate, nasal cavity and pharynx). There are also meningeal sensory branches that enter the trigeminal ganglion within the cranium. The sensory part of the mandibular nerve is composed of branches that carry general sensory information from the mucous membranes of the mouth and cheek, anterior two-thirds of the tongue, lower teeth, skin of the lower jaw, side of the head and scalp and meninges of the anterior and middle cranial fossae.

The sensory nuclei of the trigeminal nerve are located within the brainstem, in the dorsolateral pons. The mesencephalic tract and the motor nucleus of the trigeminal nerve lie more medially. The superior cerebellar peduncle lies posteriorly. It is continuous inferiorly with the spinal nucleus of the trigeminal nerve that extends into the medulla. Superiorly, the sensory nuclei on each side are continuous with the mesencephalic nucleus.

Importantly, the sensory nuclei of the trigeminal nerve receive afferent (sensory input) fibers from: (1) the trigeminal nerve ophthalmic division (e.g. general sensation from supraorbital area, cornea, iris, ethmoid sinuses), (2) trigeminal nerve maxillary division (e.g. sensation from temple, cheek, oral cavity, upper pharynx), and (3) trigeminal nerve mandibular division (e.g. sensation from middle cranial fossa, inner cheek, anterior two thirds of the tongue, chin), (4) facial nerve (e.g. general sensation from external auditory meatus), (5) glossopharyngeal nerve (e.g. general sensation from middle ear, tonsils, oropharynx, posterior one third of the tongue), (6) vagus nerve (auricular, meningeal, internal laryngeal and recurrent laryngeal branches).

Thus, primary neurons in the trigeminal ganglion synapse on the main sensory trigeminal nucleus and on the spinal trigeminal nucleus in the brainstem. The spinal nucleus of the trigeminal system extends to the upper cervical spine, where connections with cervical dermatomes exist. These dermatomes are innervated by the cervical sensory rami and the occipital nerves, which have sensory branches from C2 to C5. The trigeminal nerve also innervates stretch receptors in the muscles of mastication. The cell bodies of these neurons are in the mesencephalic trigeminal nucleus in the midbrain and pons).

### DEFINITIONS

As used herein, the words or terms set forth below have the following definitions:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition. The combination therapy disclosed herein can be locally administered by various methods. For example, intramuscular, intradermal, subcutaneous administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“Biological activity” describes the beneficial or adverse effects of a drug on living matter. When a drug is a complex chemical mixture, this activity is exerted by the substance's active ingredient but can be modified by the other constituents. Biological activity of a clostridial derivative such as a botulinum toxin can be assessed as potency or as toxicity by an in vivo LD₅₀ or ED₅₀ assay, or through an in vitro assay such as, for example, cell-based potency assays as described in U.S. 20100203559 and U.S. 20100233802.

“Botulinum toxin” means a neurotoxin produced by Clostridium botulinum, as well as a botulinum toxin, fragments, variants or chimeras thereof made recombinantly by a non-Clostridial species. The phrase “botulinum toxin”, as used herein, encompasses botulinum toxin serotype A (BoNT/A), botulinum toxin serotype B (BoNT/B), botulinum toxin serotype C (BoNT/C), botulinum toxin serotype D (BoNT/D), botulinum toxin serotype E (BoNT/E), botulinum toxin serotype F (BoNT/F), botulinum toxin serotype G (BoNT/G), botulinum toxin serotype H (BoNT/H), botulinum toxin serotype X (BoNT/X), botulinum toxin serotype En (BoNT/En), and mosaic botulinum toxins and/or their subtypes and any other types of subtypes thereof, or any re-engineered proteins, analogs, derivatives, homologs, parts, sub-parts, variants, or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“CGRP”, abbreviated for Calcitonin-Gene-Related-Peptide, as used herein encompasses any member of the calcitonin family, including any calcitonin gene related peptide and analogs, calcitonin, amylin, adrenomedullin and their analogs.

“CGRP antagonist” refers to any molecule that exhibits any one or more of the following characteristics: (a) bind to CGRP or CGRP-R and the binding results in a reduction or inhibition of CGRP activity; (b) block CGRP from binding to its receptor(s); (c) block or decrease CGRP receptor activation; (d) inhibit CGRP biological activity or downstream pathways mediated by CGRP signaling function; (e) increase clearance of CGRP; and (f) inhibit or reduce CGRP synthesis, production or release. CGRP antagonists include but are not limited to antibodies to CGRP, antibodies to the CGRP-R, small molecules that antagonize CGRP, and small molecules that antagonize CGRP-R.

“Clostridial derivative” refers to a molecule which contains any part of a clostridial toxin as defined herein. As used herein, the term “clostridial derivative” encompasses native or recombinant neurotoxins, recombinant modified toxins, fragments, chimeras and variants thereof, a Targeted Vesicular Exocytosis Modulator (TEM), or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a botulinum toxin like BoNT/A, a BoNT/B, a BoNT/Ci, a BoNT/D, a BoNT/E, a BoNT/F, a BoNT/G, BoNT/H, a BoNT/En, BoNT/X, mosaic botulinum toxins, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

“Clostridial toxin active ingredient” refers to a molecule which contains any part of a clostridial toxin that exerts an effect upon or after administration to a subject or patient. As used herein, the term “clostridial toxin active ingredient” or “clostridial derivative” encompasses a Clostridial toxin complex comprising the approximately 150-kDa Clostridial toxin and other proteins collectively called non-toxin associated proteins (NAPs), the approximately 150-kDa Clostridial toxin alone, or a modified Clostridial toxin, such as, e.g., a re-targeted Clostridial toxins.

“Combination therapy” refers to a treatment wherein a botulinum toxin and a CGRP antagonist are administered either simultaneously or sequentially, by a similar administration route or by different administration routes.

“Effective amount” as applied to the biologically active ingredient means that amount of the ingredient which is generally sufficient to effect a desired change in the subject. For example, where the desired effect is a reduction in duration or intensity of pain, an effective amount of the ingredient is that amount which causes at least a substantial reduction in duration or intensity of the pain, and without resulting in significant toxicity.

“Intramuscular” or “intramuscularly” means into or within (as in administration or injection of a CGRP antagonist into) a muscle.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Topical administration is a type of local administration in which a pharmaceutical agent is applied to a patient's skin.

“Modified botulinum toxin” means a botulinum toxin that has had at least one of its amino acids deleted, modified, or replaced, as compared to a native botulinum toxin. Additionally, s the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter, such as substance P.

“Peripheral administration” means administration by means of a peripheral location on a mammal. Peripheral administration includes subdermal, intranasal, intramuscular, intradermal, transdermal, and subcutaneous administration.

“Pharmaceutical composition” means a composition comprising an active pharmaceutical ingredient, such as, for example, a clostridial toxin active ingredient such as a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. It is also envisioned that a pharmaceutical composition comprising a Clostridial toxin active ingredient can include one or more pharmaceutically acceptable excipients that facilitate processing of an active ingredient into pharmaceutically acceptable compositions. Insofar as any pharmacologically acceptable excipient is not incompatible with the Clostridial toxin active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

“Stabilizing agent”, “stabilization agent” or “stabilizer” means a substance that acts to stabilize a Clostridial toxin active ingredient.

“Stabilizers” can include excipients, and can include protein and non-protein molecules.

“TEM” as used herein, is synonymous with “Targeted Exocytosis Modulator” or “retargeted endopeptidase.” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

“Therapeutic formulation” means a formulation can be used to treat and thereby alleviate a disorder or a disease, such as, for example, a headache or headache-associated symptoms.

“Topical administration” excludes systemic administration of the neurotoxin. In other words, and unlike conventional therapeutic transdermal methods, topical administration of botulinum toxin does not result in significant amounts, such as the majority of, the neurotoxin passing into the circulatory system of the patient.

“Treating” means to alleviate (or to eliminate) at least one symptom of a condition or disorder, such as, for example, a headache, or headache-associated symptoms, either temporarily or permanently.

“Variant” means a clostridial neurotoxin, such as wild-type botulinum toxin serotype A, B, C, D, E, F, r G, H, X, En or mosaic botulinum toxins that has been modified by the replacement, modification, addition or deletion of at least one amino acid relative to wild-type botulinum toxin, which is recognized by a target cell, internalized by the target cell, and catalytically cleaves a SNARE (SNAP (Soluble NSF Attachment Protein) Receptor) protein in the target cell.

An example of a variant neurotoxin component can comprise a variant light chain of a botulinum toxin having one or more amino acids substituted, modified, deleted and/or added. This variant light chain may have the same or better ability to prevent exocytosis, for example, the release of neurotransmitter vesicles. Additionally, the biological effect of a variant may be decreased or increased compared to the parent chemical entity. For example, a variant light chain of a botulinum toxin type A having an amino acid sequence removed may have a shorter biological persistence than that of the parent (or native) botulinum toxin type A light chain.

In some embodiments, the clostridial derivative of the present method includes a native, recombinant clostridial toxin, recombinant modified toxin, fragments thereof, targeted exocytosis modulators (TEMs), or combinations thereof. In some embodiments, the clostridial derivative is a botulinum toxin. In alternative embodiments, the clostridial derivative is a TEM.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

In some embodiments, the clostridial derivative is a botulinum toxin, which is selected from the group consisting of botulinum toxin types A, B, C₁, D, E, F, G, H, X, En and mosaic botulinum toxins. In one embodiment, the clostridial derivative of the present method is a botulinum toxin type A. The botulinum toxin can be a recombinant botulinum neurotoxin, such as botulinum toxins produced by E. coli.

The clostridial derivative, such as a botulinum toxin, for use according to the present invention can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. Acceptable excipients or stabilizers include protein excipients, such as albumin or gelatin, or the like, or non-protein excipients, including poloxamers, saccharides, polyethylene glycol, or the like. In embodiments containing albumin, the albumin can be, for example, human serum albumin or recombinant human albumin, or the like. The lyophilized material can be reconstituted with a suitable liquid such as, for example, saline, water, or the like to create a solution or composition containing the botulinum toxin to be administered to the patient.

In some embodiments, to increase the resident time of the clostridial derivative in the joint, the clostridial derivative is provided in a controlled release system comprising a polymeric matrix encapsulating the clostridial derivative, wherein fractional amount of the clostridial derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety. The therapeutically effective amount of the clostridial derivative, for example a botulinum toxin, administered according to the present method can vary according to the potency of the toxin and particular characteristics of the condition being treated, including its severity and other various patient variables including size, weight, age, and responsiveness to therapy. The potency of the toxin is expressed as a multiple of the LD₅₀ value for the mouse, one unit (U) of toxin being defined as being the equivalent amount of toxin that kills 50% of a group of 18 to 20 female Swiss-Webster mice, weighing about 20 grams each.

The therapeutically effective amount of the botulinum toxin according to the present method can vary according to the potency of a particular botulinum toxin, as commercially available botulinum toxin formulations do not have equivalent potency units. For example, one unit of BOTOX® (onabotulinumtoxinA), a botulinum toxin type A available from Allergan, Inc., has a potency unit that is approximately equal to 3 to 5 units of DYSPORT®(abobotulinumtoxinA), also a botulinum toxin type A available from Ipsen Pharmaceuticals. In some embodiments, the amount of abobotulinumtoxinA, (such as DYSPORT®), administered in the present method is about three to four times the amount of onabotulinumtoxinA (such as BOTOX®) administered, as comparative studies have suggested that one unit of onabotulinumtoxinA has a potency that is approximately equal to three to four units of abobotulinumtoxinA. MYOBLOC®, (known as NEUROBLOC® outside the United States) a botulinum toxin type B available from Elan, currently USWorldmeds , has been reported to have a much lower potency unit relative to BOTOX®. In some embodiments, the botulinum neurotoxin can be a pure toxin, devoid of complexing proteins, such as XEOMIN® (incobotulinumtoxinA). The quantity of toxin administered and the frequency of its administration will be at the discretion of the physician responsible for the treatment and will be commensurate with questions of safety and the effects produced by a particular toxin formulation. In some embodiments, the Clostridial derivative is selected from onabotulinumtoxinA, incobotulinumtoxinA, abotulinumtoxinA, daxibotulinumtoxinA, prabotulinumtoxinA, and rimabotulinumtoxinB.

Without wishing to be bound by theory a physiological mechanism can be set forth to explain the efficacy of the peripheral administration of CGRP antagonist and botulinum toxin. Peripheral administration of a combination of a CGRP antagonist and a botulinum toxin in the region of a peripheral nerve according to the methods disclosed herein is believed to permit the CGRP antagonist and botulinum toxin to either be administered to a site in the region of a patient's cranium, neck or shoulder, and/or to reduce afferent, sensory input from a site in the region of the patient's cranium, neck or shoulder, to thereby influence intracranial neurons involved in a neurological disorder, including pain and related symptoms. In addition, the combination allows for lower doses of both and/or each component. This results in decreased side effects. Furthermore, efficacy is increased by having a multimodal mechanism of action from the combination of therapeutic agents. CGRP is only one of many neuro-transmitters released by the peripheral nerves and botulinum toxins have the ability to block more than CGRP release. CGRP antagonists will have enhanced action on pain alleviation, by combination with botulinum toxins as these will block other neurotransmitters such as substance P and glutamate.

Administration in the region of a peripheral nerve, a cranial nerve, or combination thereof, including but not limited to a trigeminal, occipital, autonomic, spinal or cervical sensory nerve(s) of a CGRP antagonist in combination with a botulinum toxin in accordance with the present disclosure can also block progression of neurological disorders, including headache, migraine and related symptoms mediated by repeated sensory input to the cortex from a sensory nerve and also from autonomic nervous system components.

Methods and medicaments for treating, preventing, alleviating or reducing the frequency of occurrence of pain according to the present disclosure can comprise a CGRP antagonist in combination with a clostridial derivative, for example, a botulinum toxin, for peripherally administration in the region of a peripheral nerve of a patient. The CGRP antagonist is administered in a therapeutically effective amount to alleviate pain.

In some embodiments, the disclosure provides for the peripheral administration in the region of a peripheral nerve, of a combination of CGRP antagonists and optionally a clostridial derivative, for example a botulinum toxin, to treat (including alleviate and/or prevent) a variety of CSD and pain related disorders.

In some embodiments, the clostridial derivative, for example a botulinum toxin, is administered to a trigeminal nerve. Trigeminal nerve administration of botulinum toxins has been disclosed for example in U.S. Pat. Nos. 8,609,112; 8,609,113; 8,734,810; 8,717,572; 9,238,061 and 10,064,921; each of which is hereby incorporated by reference in its entirety.

In some embodiments, the clostridial derivative, for example a botulinum toxin, is administered to a suture line. Suture line administration of botulinum toxins has been disclosed for example in U.S. Pat. Nos. 8,617,571; 9,248,168; 9,827,297; and 10,220,079; each of which is hereby incorporated by reference in its entirety.

### EXAMPLES

The following non-limiting examples provide those of ordinary skill in the art with possible case scenarios and specific methods to treat conditions within the scope of the present disclosure and are not intended to limit the scope of the disclosure. In the following examples administration of a CGRP antagonist in combination with a botulinum toxin can be carried out. For example, by topical application (cream or transdermal patch), subcutaneous injection, or subdermal implantation of a controlled release implant.

### Example 1

### Peripheral Administration of an Exemplary Clostridial Derivative to Alleviate Pain

A 46 year old woman presents with pain localized at the deltoid region due to an arthritic condition. The muscle is not in spasm, nor does it exhibit a hypertonic condition. The patient can be treated with erenumab (70 mg or 140 mg, subcutaneous, once monthly) followed by treatment with a bolus injection of between about 50 Units and 200 units of intramuscular onabotulinumtoxinA into the deltoid muscle and surrounding muscles. The botulinum toxin administration can be within one month of the subcutaneous administration with erenumab. Within 1-7 days after neurotoxin administration the patient's pain is substantially alleviated. The duration of significant pain alleviation can be from about one to about six months. A pain in the shoulder, arm, and hand due to osteoporosis, fixation of joints, coronary insufficiency, cervical osteoarthritis, localized shoulder disease, or due to a prolonged period of bed rest can be similarly treated.

### Example 2

A 43-year-old female has fibromyalgia. The patient walks with a normal gait and is able to perform heel walk and toe walk without difficulty. Range of motion of the lumbar and cervical spine is complete but painful in extremes of range. Motor and sensory examination of the upper and lower extremities reveals mild S1 sensory loss on the right side. Reflexes are intact at the knees and ankles. Straight leg raise is negative. Further examination reveals the presence of multiple tender points in the occipital, lower cervical, trapezium, gluteal, and greater trochanter areas bilaterally.

She then starts treatment with atogepant at a dose of 60 mg b.i.d. After a week of treatment with atogepant, the patient is treated with subcutaneous injections of 60 units of onabotulinumtoxinA approximately 5 centimeters from the occipital, lower cervical, trapezius, gluteal, and greater trochanter tender points. After approximately one week the patient reports herself to be pain free.

1. A method for treating, preventing, alleviating or reducing the frequency of occurrence of pain in a patient in need thereof, comprising administering to the patient an antagonist of calcitonin gene-related peptide (CGRP-antagonist), wherein said patient is concurrently undergoing treatment with a clostridial derivative; wherein said pain is other than pain associated with headache or migraine.

2. A method for treating, preventing, alleviating or reducing the frequency of occurrence of pain in a patient in need thereof, comprising administering to the patient:
(a) an antagonist of calcitonin gene-related peptide (CGRP-antagonist); and,
(b) a clostridial derivative;
wherein said pain is other than pain associated with headache or migraine

3. The method according to claim 2 wherein said pain is neuropathic type pain, or
wherein said pain is inflammatory type pain, or
wherein said pain is selected from pain caused by diabetes mellitus type I or II, viral or retroviral infection, cancer chemotherapy, radiotherapy, a surgical procedure, alcoholism, facial neuralgia, trauma, radiculopathy or radiculagia, cruralgia or thoracic outlet syndrome, fibromyalgia and restless leg syndrome; or
wherein said pain is selected from acute and chronic inflammatory demyelinating polyradiculoneuropathy; alcoholic polyneuropathy; chemotherapy-induced polyneuropathy; complex regional pain syndrome; an entrapment neuropathy; HIV sensory neuropathy; an iatrogenic neuralgia; idiopathic sensory neuropathy; nerve compression or infiltration by a tumor; nutritional deficiency-related neuropathies; painful diabetic neuropathy; phantom limb pain; postherpetic neuralgia; postradiation plexopathy; radiculopathy; toxic exposure-related neuropathies; tic douloureux; and/or posttraumatic neuralgias; or
wherein said pain is selected from post-surgical pain, post-operative pain, dental pain, trigeminal neuralgia, pain associated with burn, wound or kidney stone, pain associated with trauma, traumatic head injury, pain associated with musculo-skeletal disorders, rheumatoid arthritis, osteoarthritis, visceral pain, colitis, pancreatitis, gastritis, ankylosing spondylitis, sero-negative (non-rheumatoid) arthropathies, non-articular rheumatism and peri-articular disorders, and pain associated with cancer, pain associated with sickle-cell anemia, peripheral neuropathy, post-herpetic neuralgia, herpetic neuralgia, general neuralgia, postherpetic neuralgia; or
wherein said pain is selected from rheumatic pain, pain associated with mucositis, and dysmenorrhea, post-surgical pain and/or cancer pain, pain associated with rheumatoid arthritis or pain associated with osteoarthritis; or
wherein said pain is associated with diabetic vasculopathy, diabetic retinopathy or diabetic symptoms associated with insulitis; or
wherein said pain is acute pain; or
wherein said pain is chronic pain; or
wherein said pain is nociceptive pain, pain associated with multiple sclerosis, pain associated with irritable bowel syndrome or inflammatory bowel disease, pain associated with dysmenorrhea, pelvic pain, pain associated with cystitis, pain associated with pancreatitis, pain associated with Crohn's disease, pain associated with epilepsy or an epileptic condition, radicular pain, sciatica, back pain, head or neck pain, severe or intractable pain, breakthrough pain, postsurgical pain, stroke, cancer pain, seizure disorder, causalgia, chemo-induced pain and anxiety, bladder pain, liver pain and pancreatic pain.

4-6. (canceled)

7. The method according to claim 2 wherein said clostridial derivative is botulinum toxin of immunotype A, B, C, D, E, F, or G.

8. The method according to claim 7 wherein the botulinum toxin is onabotulinumtoxinA.

9. The method according to claim 2 wherein said CGRP-antagonist is an anti-calcitonin gene-related peptide receptor antibody selected from the group consisting of galcanezumab, fremanezumab, eptinezumab, and erenumab.

10. (canceled)

11. The method according to claim 1 wherein said antagonist of CGRP receptor is selected from ubrogepant, atogepant, rimegepant or a pharmaceutically acceptable salt thereof.

12-18. (canceled)

19. The method according to claim 9 wherein said anti-CGRP antibody is administered at a dosage that is about 20% or 30% or 40% or 50% or 60% or 70% or 80% lower than the recommended dosage for the anti-CGRP antibody monotherapy.

20. The method according to claim 2 wherein said botulinum toxin is administered once every four weeks, five weeks, six weeks, seven weeks, eight weeks, nine weeks or ten weeks.

21. The method according to claim 2 wherein said clostridial derivative is administered to a peripheral nerve, a cranial nerve, or combinations thereof.

22. The method according to claim 9 wherein said anti-calcitonin gene-related peptide receptor antibody is administered to a peripheral nerve, a cranial nerve, or combinations thereof.

23. The method according to claim 2 wherein said patient is administered one or more additional medications for the treatment of pain, wherein said additional medication is selected from morphine, codeine, hydrocodone, oxycodone, fentanyl, pethidine, methadone, pentazocine, sufentanil, levorphanol, dihydrocodeine, nalbuphine, butorphanol, tramadol, meptazinol, buprenorphine, dipipanone, alfentanil, remifentanil, oxymorphone, tapentadol, propoxyphene, and hydromorphone.

24. (canceled)

25. The method according to claim 2 wherein said patient is administered one or more additional medications for the treatment of pain, and wherein said one or more additional medications is selected acetaminophen, ibuprofen, ketaprofen, naproxen, aspirin and pharmaceutically acceptable salts, esters, conjugates, or prodrugs thereof.

26. The method according to claim 9 wherein said anti-CGRP antibody is erenumab, and wherein erenumab is administered subcutaneously at a dose of about 5 mg to about 500 mg every one, two, three, four, five, six, seven, eight, nine or ten weeks.

27-39. (canceled)

40. The method according to claim 9 wherein said anti-CGRP antibody is galcanezumab, and wherein galcanezumab is administered subcutaneously at a dose of about 10 mg to about 500 mg every one, two, three, four, five, six, seven, eight, nine or ten weeks.

41-53. (canceled)

54. The method according to claim 9 wherein said anti-CGRP antibody is fremanezumab, and wherein fremanezumab is administered subcutaneously at a dose of about 100 mg to about 1000 mg every one, two, three, four, five, six, seven, eight, nine or ten weeks.

55-70. (canceled)

71. The method according to claim 9 wherein said anti-CGRP antibody is eptinezumab, and wherein eptinezumab is administered subcutaneously at a dose of about 50 mg to about 1000 mg every one, two, three, four, five, six, seven, eight, nine or ten weeks.

72-85. (canceled)

86. The method according to claim 2 wherein said onabotulinumtoxinA is administered at a dose of about 1 unit, about 2 units, about 3 units, about 4 units, about 5 units, about 6 units, about 7 units, about 8 units, about 9 units or about 10 units every one, two, three, four, five, six, seven, eight, nine, ten, eleven, twelve, thirteen, fourteen, fifteen or sixteen weeks; or
wherein said onabotulinumtoxinA is administered at a dose of about 10 unit, about 15 units, about 20 units, about 25 units, about 30 units, about 40 units, about 45 units, about 50 units, about 55 units or about 60 units every one, two, three, four, five, six, seven, eight, nine, ten, eleven, twelve, thirteen, fourteen, fifteen or sixteen weeks; or
wherein said onabotulinumtoxinA is administered at a dose of about 25 unit, about 50 units, about 75 units, about 100 units, about 125 units, about 150 units, about 175 units, about 200 units, about 225 units or about 250 units every one, two, three, four, five, six, seven, eight, nine, ten, eleven, twelve, thirteen, fourteen, fifteen or sixteen weeks; or
wherein said onabotulinumtoxinA is administered at a dose of about 1 to about 1,000 units every one, two, three, four, five, six, seven, eight, nine, ten, eleven, twelve, thirteen, fourteen, fifteen or sixteen weeks; or
wherein said onabotulinumtoxinA is administered at a dose of about 1 to about 100 units every one, two, three, four, five, six, seven, eight, nine, ten, eleven, twelve, thirteen, fourteen, fifteen or sixteen weeks; or
wherein said onabotulinumtoxinA is administered at a dose of about 10 to about 50 units every one, two, three, four, five, six, seven, eight, nine, ten, eleven, twelve, thirteen, fourteen, fifteen or sixteen weeks.

87-97. (canceled)

98. The method according to claim 2 wherein said antagonist of CGRP receptor is ubrogepant or a pharmaceutically acceptable salt thereof, and ubrogepant is administered at a dose of about 5 to about 500 mg per day.

99. (canceled)

100. The method according to claim 2 wherein said antagonist of CGRP receptor is atogepant or a pharmaceutically acceptable salt thereof, and atogepant is administered at a dose of about 5 to about 500 mg per day.

101. (canceled)
